(19) United States
(12) Patent Application Publication
(10) Pub. No.: US 2010/0280526 A1
(43) Pub. Date: Nov. 4, 2010

(54) Medical Device With Articulating Shaft Mechanism

(75) Inventors: Thomas Weisel, Ventura, CA (US); Roger Pisarnwongs, Valencia, CA (US); David M. Skinlo, North Logan, UT (US)

Correspondence Address: KOPPEL, PATRICK, HEYBL & DAWSON, 2815 Townsgate Road, SUITE 215, Westlake Village, CA, 91361-5827, US

(73) Assignee: ARCH DAY DESIGN, LLC

(21) Appl. No.: 12/769,040

(22) Filed: Apr. 28, 2010

Related U.S. Application Data
(60) Provisional application No. 61/173,769, filed on Apr. 29, 2009.

(51) Int. Cl. A61B 19/00

(52) U.S. Cl. 606/130

(57) ABSTRACT

A medical device with an articulating shaft assembly includes at least one slat member which has a cross section that allows it to be substantially flexible in a first plane and relatively stiff in the plane perpendicular to the first plane. A bendable section located at the working end of the device includes at least a portion of one or more slat members, such that the bendable section will articulate in the first plane in response to tensive and/or compressive forces applied to the bendable section. The device also includes a shaft section which is coupled to and supports the bendable section, and at least one control mechanism arranged to cause the tensive and/or compressive forces needed to articulate the bendable section to be applied to the bendable section.

### RELATED APPLICATIONS

This application claims the benefit of provisional patent application No. 61/173,769 to T. Weisel et al., filed Apr. 29, 2009.

### BACKGROUND OF THE INVENTION

1. Field of the Invention

This invention relates generally to hand-held medical devices, and more particularly to an articulating shaft mechanism for a hand-held medical device.

2. Description of the Related Art

The medical device industry is constantly striving to produce instruments which enable surgical procedures to be less invasive, such that the affected areas can be accessed while the surrounding tissues are virtually unaffected. This is particularly important when performing arthroscopic procedures, for example, due to their characteristically small incisions. Due to the small incision size, the effective area which can be safely accessed with a traditional straight-shafted device such as a forceps or grasper will be limited.

Another key consideration with respect to medical devices is that of cost. Various instruments have been developed to increase the effective area which can be safely accessed; however, such instruments tend to be complex and costly. Since both cost and effectiveness are typically taken into account when selecting a medical device, these more complicated instruments often prove to be unacceptable.

### SUMMARY OF THE INVENTION

The present invention is an enabling medical device shaft technology which provides a larger effective range of operation than conventional devices, while maintaining the stiffness and functionality needed in practice. One application of this technology is to the shaft of hand-held medical devices, such as forceps, graspers, or needle delivery devices, which benefit from this technology by allowing greater access to affected areas during traditional arthroscopic surgery.

The present instrument shaft technology provides a medical device with an articulating shaft assembly. The device includes at least one slat member (also referred to herein as simply a ‘slat’), with each slat member having a predetermined cross section which allows it to be substantially flexible in a first plane and relatively stiff in the plane perpendicular to the first plane. A ‘bendable’ section is located at the distal ‘working end’ of the device, and at least a portion of at least one slat member runs through the bendable section. The slat members are typically fixed to the distal end of the bendable section, such that the bendable section will articulate in the first plane in response to tensive and/or compressive forces applied to the bendable section. The device also includes a shaft section which is coupled to the bendable section and is sufficiently stiff to support the bendable section, and at least one control mechanism arranged to cause the tensive and/or compressive forces needed to articulate the bendable section to be applied to the bendable section. These features combine to form a device which offers performance equivalent to that of conventional straight-shafted devices, but with a larger effective range of operation.

These and other features, aspects, and advantages of the present invention will become better understood with reference to the following drawings, description, and claims.

### BRIEF DESCRIPTION OF THE DRAWINGS

FIG. 1 is an isomeric view of the articulating shaft of a medical device per the present invention.

FIG. 2 is a detailed view of the distal end of the shaft shown in FIG. 1, showing both straight (neutral) and bent (active) positions.

FIG. 3 is a detailed view of the proximal end of the shaft shown in FIG. 1.

FIGS. 4-6 are top views of the articulating shaft of a medical device per the present invention, showing the effect of the slat members in various positions.

FIG. 7 is an isometric view of a slat member and a wire/cable actuator element.

FIG. 8 is a detailed isometric view of wire/cable and slat member actuator elements.

FIG. 9 is a sectional view of the bendable portion of a shaft per the present invention, which includes multiple slat member channels.

FIG. 10 is a sectional view of the bendable portion of a shaft per the present invention, which includes a single slat member channel.

FIG. 11 is a sectional view of the bendable portion of a shaft per the present invention, shown in the bent position.

FIG. 12 is a sectional view of the bendable portion of a shaft per the present invention, which includes small notches for improved flexibility.

FIGS. 13-17 are detailed cross-sections of respective bendable sections illustrating several possible slat member and working channel layouts.

FIGS. 18a and 18b are a sectional and close-up views, respectively, of one possible termination assembly for a shaft per the present invention.

FIG. 19 is an isometric view of a shaft assembly showing an alternative slat member termination assembly.

FIG. 20 is a detailed isometric view of an alternative slat member termination assembly.

FIG. 21 is a detailed isometric view of a slat termination feature.

FIG. 22 shows side views of a slat, illustrating two options for slat member termination.

FIG. 23 is an isometric view of bendable section design employing a spring.

FIG. 24 is a detailed isometric view of the bendable section design of FIG. 23.

FIG. 25 is an exploded view of a shaft assembly per the present invention in which the bendable section is comprised of two materials of different durometers.

FIGS. 26a and 26b are detailed views of the shaft assembly of FIG. 25.

FIG. 27 is an isometric view of a split bendable section design.

FIG. 28 is a detailed isometric view of the split bendable section of FIG. 27.

FIG. 29 is a top view of a shaft assembly per the present invention, highlighting the motion and counter-motion of the distal and proximal ends of the assembly.

FIG. 30 is a detailed view of a handle assembly that might be used with a shaft assembly in accordance with the present invention.

FIG. 31 is an embodiment of a shaft assembly per the present invention which includes a sensor.

FIG. 32 is an embodiment of a shaft assembly per the present invention which includes an opening for transmitting light or radiation.

FIG. 33 is an embodiment of a shaft assembly per the present invention which includes a jaw for retraction or gripping.

FIG. 34 is an embodiment of a shaft assembly per the present invention which includes a scissor or alternative cutting device.

FIG. 35 is an elevation view of a handle assembly that might be used with a shaft assembly in accordance with the present invention.

FIG. 36 is a sectional view of the handle assembly of FIG. 35.

FIG. 37 is an elevation view of the handle assembly of FIG. 35, illustrating the operation of the handle assembly.

### DETAILED DESCRIPTION OF THE INVENTION

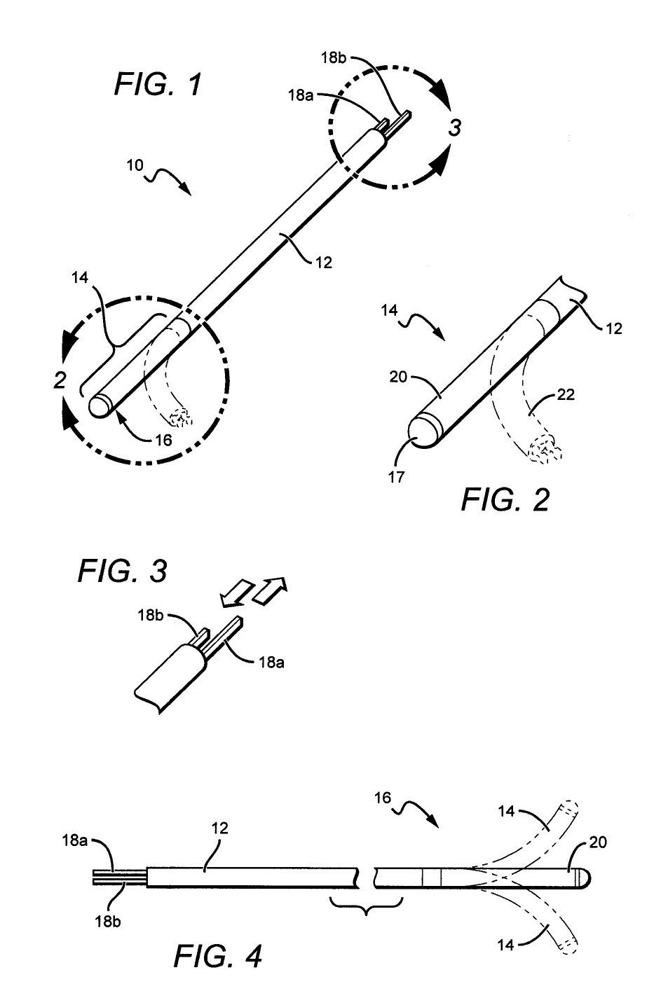

The present medical device includes an articulating shaft, which enables the device to provide a larger effective range of operation than conventional instruments. FIG. 1 illustrates the basic elements of an articulated shaft assembly 10 in accordance with the present invention; additional details of the shaft assembly are shown in FIGS. 2 and 3. The assembly includes a shaft section 12 coupled to a bendable section 14 at the distal working end 16 of the device which terminates at a tip 17; the shaft section is arranged to be sufficiently stiff to support the bendable section. The assembly also includes at least one slat member 18a; a second slat, 18b, is also shown, though the invention can be practiced with as little as one slat. Each slat has a predetermined cross section which allows the slat member to be substantially flexible in a first plane and relatively stiff in the plane perpendicular to the first plane. At least one slat member extends into bendable section 14. The slat members are typically fixed to the distal end of the bendable section and arranged such that, when the bendable section is subjected to tensive and/or compressive forces, the slats allow the bendable section to articulate in the first plane, but not in the perpendicular plane.

Tensive and/or compressive forces can be applied to the bendable section in several ways. For example, two opposed slat members can be fixed at one end to the distal end of the bendable section, and the bendable section articulated based on the counter-motion of the opposed slats. Alternatively, one or more stationary slat members can be employed—i.e., slats which are fixed at both ends, with one end fixed to the distal end of the bendable section—along with a movable wire, cable and/or slat which is fixed at one end to the distal end of the bendable section. Here, the bendable section is articulated in the first plane by moving the wire, cable or slat with respect to the stationary slat members. The reverse configuration is also valid, such that the wire or cable act as the stationary member and the slat is movable.

It should be noted that only one slat member is needed to provide stiffness in the plane perpendicular to the first plane; articulation can be performed with the aid of another slat member and/or a non-slat element such as a wire or cable—running alongside the first slat. At least one control mechanism (not shown) is typically attached to the proximal end of the shaft assembly, opposite the working end, which is operated to move the slat members and/or non-slat element and thereby apply the tensive and/or compressive forces needed to cause the bendable section to articulate.

Shaft section 12 provides the support and length necessary to enable bendable section 14 to fully access the space needed. Bendable section 14 is coupled directly to one end of shaft section 12, with slat members 18a, 18b threaded directly through the shaft and bendable sections.

When the control mechanism is not imparting any force to cause bendable section 14 to articulate, the bendable section is said to be in a ‘neutral’ state. When the control mechanism is imparting force to cause bendable section 14 to articulate, the bendable section is in an ‘active’ state. Bendable section 14 is typically in-line with shaft section 12 when in the neutral state and bent with respect to the shaft section when in the active state; this is illustrated in FIG. 2 (neutral state 20; active state 22). However, a device per the present invention might alternatively be arranged such that bendable section 14 is pre-bent, such that it is bent at a first angle with respect to the shaft section in the neutral state and is in-line with the shaft section or at an angle other than the first angle when in the active state.

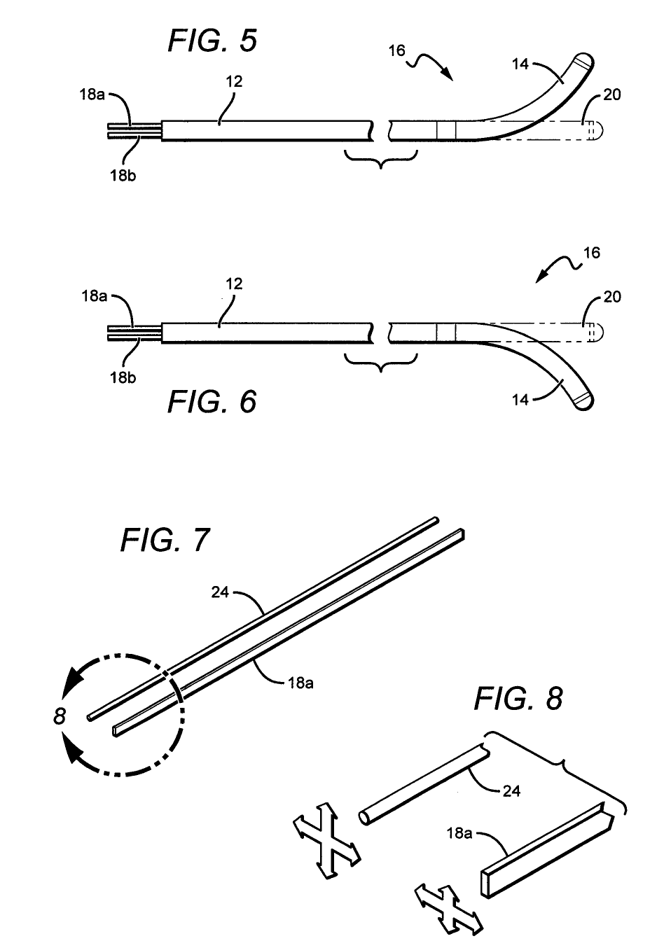

As shown in FIG. 4, articulation of the bendable section can be based on the counter-motion of opposed slats 18a and 18b. As can be seen in FIG. 5, as slat 18a is pulled and slat 18b is pushed or compressed, the working end 16 is manipulated as shown. By reversing the action of the slats, the working end 16 is manipulated as shown in FIG. 6.

A distinguishing element of the present device is the use of slat members such as 18a and 18b to create the articulating action, as compared to wires or cables 24 alone as are used in traditional articulating devices (FIG. 7). Slat members can be pulled in tension but, unlike wires or cables, can also be pushed in compression. This allows for enhanced manipulation and also fixes the bendable section's position better in comparison with a wire or cable system, because the number of forces that can be applied is doubled (tension on one side and compression on the other side of the neutral axis).

FIG. 8 illustrates the relative compliance of a slat member 18a vs. wire/cable 24. Wire/cable 24 has compliance in two perpendicular planes, with the relative flexibility of each of these planes being generally equivalent. By comparison, slat member 18a has a biased flexibility, with one plane being high in flexibility and the perpendicular plane remaining relatively stiff. This bias is a function of the slat member's relative thickness-to-height ratio, which can be adjusted as needed (but is preferably at least two-to-one). In order to have the required degree of flexibility, the slat members are preferably constructed of a super-elastic material—preferably Nitinol—which would allow for the required flexibility and durability during repeated cycling. The slat members might alternatively be constructed from a shape-memory material, or with other materials having the desired characteristics, such as stainless steel and plastics, selected based on the design requirements.

It should also be noted that, with low thickness-to-height ratios, the cross-sectional area of a slat member—and thus the compressive and tensile load that the slat can withstand—can be much greater than that of an equally flexible (in one plane) wire/cable. A slat member's resistance to compressive loads is primarily a function of its column strength, which generally resists compression better than cables or wires. This combination of features allows a generally flexible element to provide the necessary support for articulation, as well as for the other forces generated during cutting and/or manipulation.

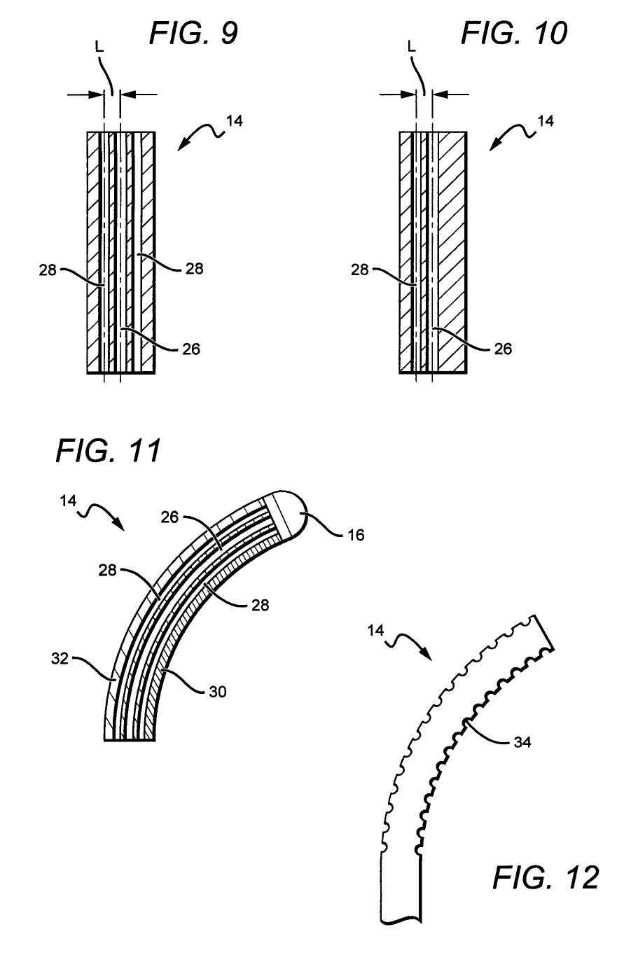

Some form of support and guidance means is preferably employed to ensure proper functioning of the slat members. One exemplary means comprises an extruded tube which has ‘slat channels’ defined just off of the shaft centerline for holding and guiding the slat members through the bendable section; an additional ‘working channel’ which traverses the bendable section can then be directed generally down the centerline if needed. This configuration can take on a number of forms depending on the application, but in general includes at least one slat channel and at least one working channel. A cross-section of a bendable section 14 which includes a working channel 26 and two slat channels 28 is shown in FIG. 9; this is a preferred embodiment. An embodiment employing a working channel 26 and a single slat channel 2—which can be useful in applications in which space constraints limit the size of bendable section 14—is shown in FIG. 10. Embodiments which include no working channels are also contemplated; such an arrangement could be used, for example, as a curvable measuring stick.

The geometries of bendable section 14 play a critical role in the basic function of the articulation mechanism. For example, referring to FIGS. 9 and 10, the length L between the centerline of a slat channel 28 and working channel 26 is one factor that affects the amount of torque created about the centerline of the shaft, which causes the shaft to bend as shown in FIG. 11. Embodiments in which the slats are located in the bendable section only are also contemplated. In this case, slat members are used to make the bendable section relatively stiff in one plane and flexible in the perpendicular plane, and a wire or some other actuation method is used to cause the bendable section to be articulated.

For the embodiment shown in FIG. 11 which employs two opposed slat members, bendable section 14 begins to articulate as torque is applied by the slat members. This articulation is not only created by the torque of the slats, but also by the compression 30 and the stretch 32 of the walls of bendable section 14. This mechanism can be adjusted by utilizing materials of different geometries and durometers. It is also noted that materials can be selected and the bendable section designed in a manner which allows the creation of folds or ripples in the section, to achieve the same desired results.

Another alternative would be to remove or relieve material to allow for additional flexibility in certain sections of the shaft. FIG. 12 illustrates this concept with the creation of small notches 34 that have been removed from the sides of bendable section 14.

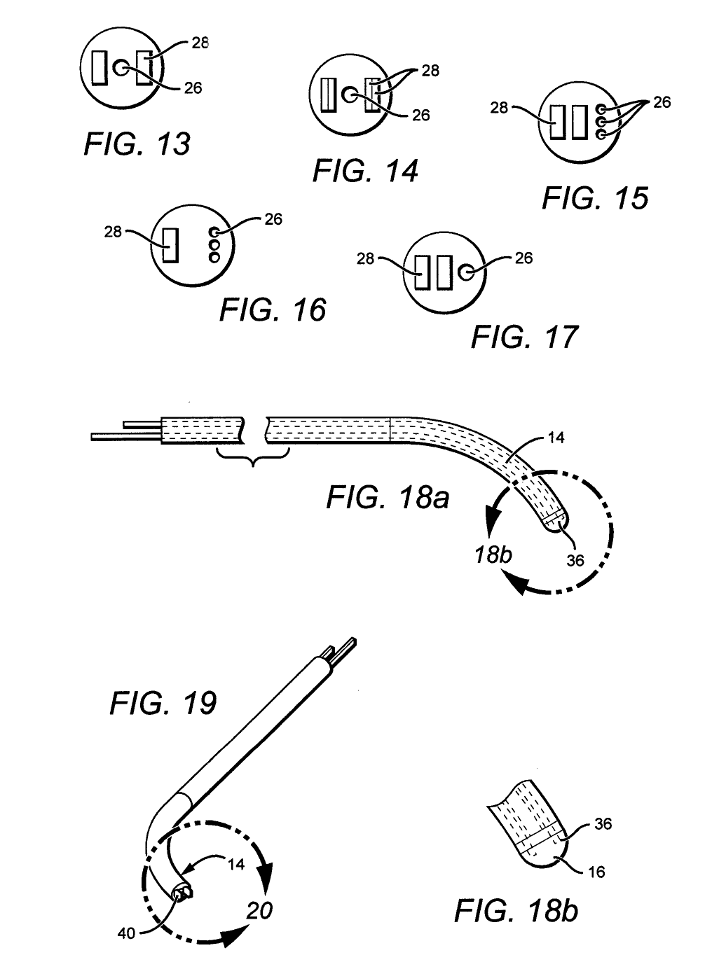

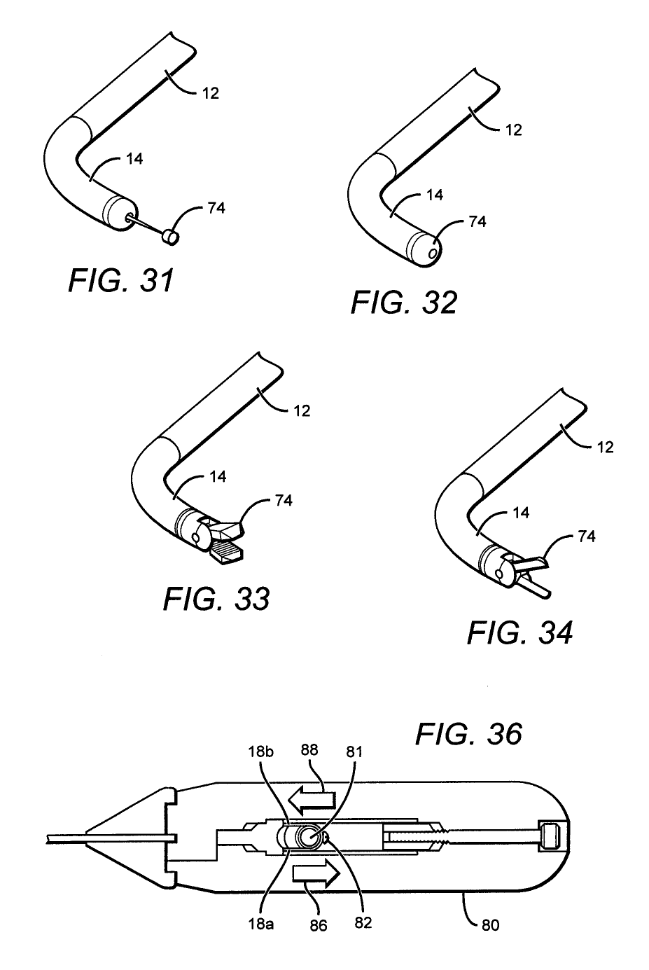

As can be seen in the examples shown in FIGS. 13-17, numerous other configurations of slat and working channels are possible, including those with a single slat channel and multiple working channels. The layout of these slat and working channels would typically be selected based on the needs of a particular application and the number of features to be delivered to the tip of the articulating shaft. These features, referred to herein as ‘end effectors’, could include, for example, sensors, cutting devices such as a scissors, a pair of jaws for retraction or gripping, a light or radiation source, a fluid delivery means, ablation devices, burrs and suture passing devices (see, e.g., FIGS. 31-34). An end effector could be coupled to, for example, one or more slat members which pass through the bendable section, which could be pulled or pushed as needed to operate and/or manipulate the effector. Note that a medical device per the present invention might also include one or more wires which pass through the shaft assembly via respective channels, which can be pushed (assuming adequate stiffness) or pulled to operate and/or manipulate an end effector. A combination of slat members and wires could also be employed to manipulate an end effector.

Material selection is another key aspect of the design of bendable section 14. Preferably, the bendable section is constructed of an extruded or molded tube. This extrusion can be composed of plastics such as silicone, thermoplastic elastomers (TPE), polyurethane, and/or rubbers or plastics.

In order for the bendable section to function with the slat assembly, it is critical that they be combined in such a way as to control the relative movement of each component, allowing each component to move or to remain fixed as necessary for the design. FIGS. 18a, 18b and 22 illustrate one method for fixing the terminal end of the slats to the tip or distal end of bendable section 14. This securing means utilizes features such as grooves, notches 36 or holes 38 at the terminal end of the slats to provide secure fixation to the tip of working end 16. These features might form a friction-type fit, as with a notched design, or a mechanical lock between the components, as with a pin-type securing mechanism such as holes 38. Each of these approaches relies on a feature smaller in height than the overall height of the slat.

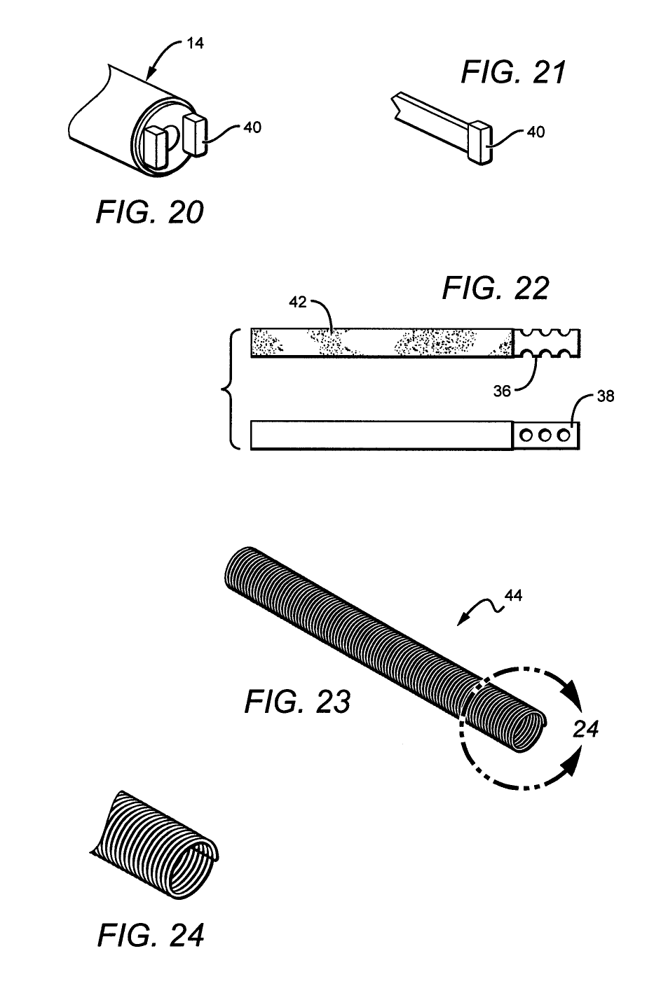

An alternative approach, shown in FIGS. 19, 20 and 21, requires terminating each slat member with a feature which is larger than the slat height. This protrusion works to keep the slat member from retracting into bendable section 14. Termination 40 can be fully captured by attaching a tip to the body of the device (not shown) which restricts movement during compressive slat forces.

The slat members are preferably coated with a lubricous material (42 in FIG. 22), which would keep them from sticking to the bendable section during both manufacturing and operation. The slat channels would preferably be coated with a Parylene or TFE-type coating or coated with an oil, to reduce or eliminate adhesion of the slat member(s).

One possible alternative to constructing the bendable section completely out of plastics might be to use a coiled spring 44 as the bendable section, as shown in FIGS. 23-24. This would provide a large degree of flexibility, while allowing an increased resistance to compressive loads which would be working to collapse the bendable section. The slat member or members would be attached to the distal end of the spring only.

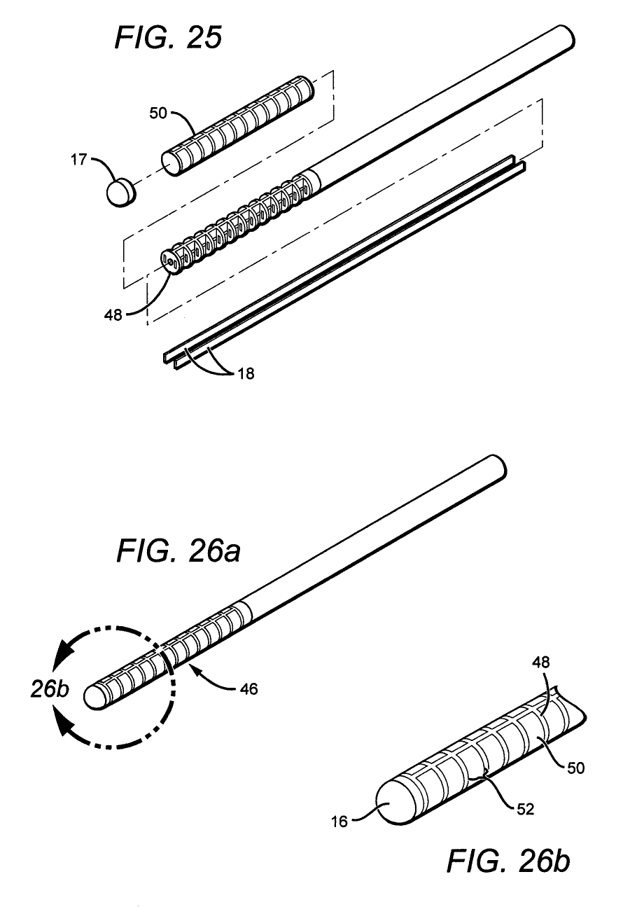

The bendable section might also be a co-extrusion, which would be constructed of materials having various durometers. FIG. 25 is an exploded view and FIGS. 26a and 26b assembled views of a dual durometer co-extrusion embodiment. The overall structure of the shaft assembly is similar to previous embodiments with the exception of the bendable section 46. Here, the bendable section is comprised of two materials of different durometers, one relatively high (48) and one relatively low (50). High durometer material 48 creates a type of backbone, which supports and guides the slat members and working channels while at the same time allowing low durometer materials 50 to fill the interstitial spaces of the backbone. As the assembly is articulated, the low durometer materials 50 compress or displace, collapsing spaces 52 and allowing the device to move easily in the selected direction. On the opposite side of the backbone, material 50 will stretch—allowing the size of spaces 52 to increase.

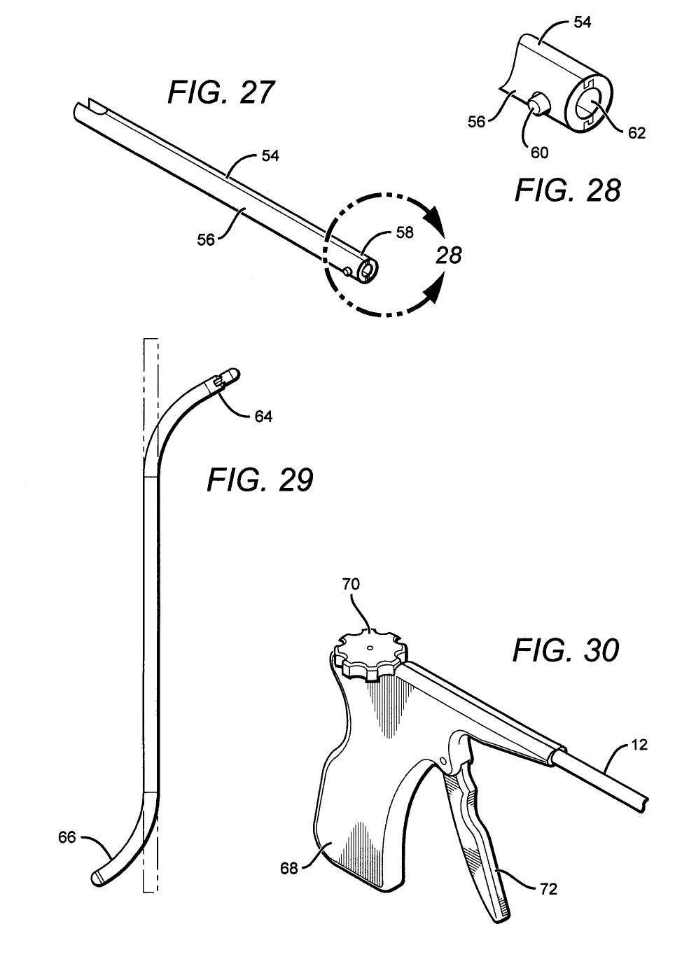

Another possible embodiment requires providing a slat assembly that also serves as the outer wall of the bendable section. FIGS. 27-28 illustrate such a bendable section made from two half section components 54, 56 which interlock longitudinally to form a tube. This slat/tube design provides the same push-pull actuation mechanism described above, but without the requirement for differentiated slat members; i.e., for this embodiment, the half sections function as the slat members. As components 54 and 56 are moved in opposition to one another, the tip (58) of the assembly would be caused to articulate. The articulation is created by the counter-motion of the halves 54, 56 and the fact that the tip of each shaft-half is pinned (60) or fixed relative to one another. This design would also allow for a working channel 62 to pass previously described features such as lights, grippers, etc. to the distal/working end of the device.

FIG. 29 illustrates a top view of one possible embodiment of a medical device with an articulating shaft in accordance with the present invention. Here, the articulation force at the distal tip 64 is created by applying a similar displacement to a bendable section 66 at the proximal end of the device, which will be mirrored in the distal tip 64 via one or more slat members (not shown) as described above.

The displacement of bending section 66 at the proximal end can be achieved with a variety of mechanisms; one possible mechanism is illustrated in FIG. 30. This exemplary mechanism highlights the general ergonomics of a preferred embodiment, in that it includes a main handle portion 68, an actuating means 70, and an additional lever or mechanism 72 for controlling features 74 at the working end of the device such as jaws, scissors, etc., examples of which are shown in FIGS. 31-34.

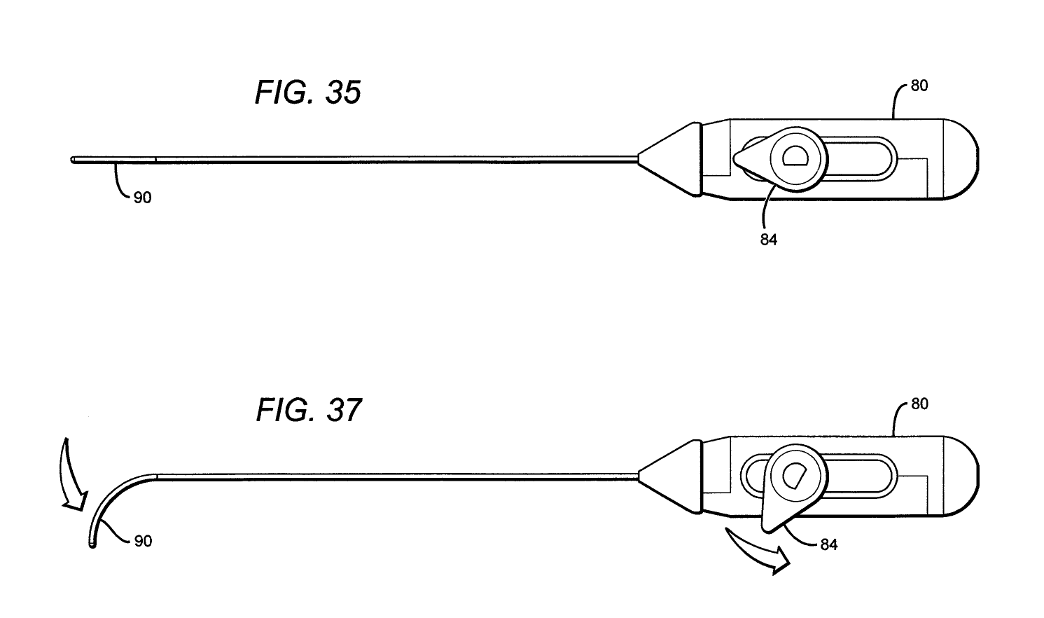

One possible implementation of a handle mechanism which can be used to control the bendable section of a device employing two opposed slat members is shown in FIGS. 35-37. Here, a screwdriver type handle 80 receives the proximal end of opposed slats 18a and 18b, which are coupled to an actuation means 81. It should be noted that the slats 18a and 18b can alternatively be a single continuous piece as shown in FIG. 36. Also in FIG. 36, actuating means 81 is a rotating shaft, to which slats 18a and 18b are attached via a screw 82. The rotating shaft is attached to a thumb lever 84 which extends outside of handle 80. As shown in FIGS. 36 and 37, rotation of thumb lever 84 in the counter-clockwise direction results in a pulling action 86 on slat 18a and a pushing action 88 on slat 18b, which causes the bendable section 90 to articulate. Note that the bendable section may also be articulated in the opposite direction by rotating thumb lever 84 in the clockwise direction.

The present shaft assembly can benefit from several possible manufacturing options, such as molding or over-molding including multi-shot. A wide variety of materials can also be used as needed to achieve the required durability and flexibility. Note that the tube or structure that houses the slat or slats does not have to have a uniform cross-section along the length of the bendable section. For instance, if the tube diameter tapers smaller as it nears the distal end, the bend radius may be more uniform due to the distribution of stresses.

A very common procedure which might benefit from a device made per the present invention is a meniscectomy, which is performed to remove small pieces of loose tissue from the meniscus of the knee. This is traditionally completed through a single portal, with an additional portal being utilized for visualization, via a scope. Traditional instruments have a generally straight shape which makes it difficult to reach the anterior horn of the meniscus. This lack of access may require the surgeon to change instruments or leave the anterior portion of the horn untreated. If a device with an articulating shaft as described herein were employed instead, the entire length of the meniscus might be treated with a single instrument, due to the ability of the device's working end to articulate and thereby reach the full arc of tissue. There are a number of anatomies and treatments which could benefit from this increased access, including knees, hips, endoscopic and spinal treatments.

In summary, the features of the present invention combine to provide a medical device which has performance equivalent to that of conventional/straight devices, but with a larger working range or greater access. This, coupled with design elements which are simple and less expensive to manufacture, combine to form a device which constitutes a significant improvement over the current industry standards.

It is noted that the descriptions and embodiments describe herein are not exhaustive and it is appreciated that there are many adjustments and modifications that might be included which achieve substantial similar results. The intended embodiments and scope are more explicitly defined in the following claims.

We claim:

1. A medical device with an articulating shaft assembly, comprising:
at least one slat member, each of said slat members having a predetermined cross section which allows the slat member to be substantially flexible in a first plane and relatively stiff in the plane perpendicular to said first plane;
a bendable section located at the working end of said device, said bendable section including at least a portion of at least one of said slat members and arranged to articulate in said first plane in response to the application of tensive and/or compressive forces;
a shaft section which is coupled to said bendable section and is sufficiently stiff to support said bendable section; and
at least one control mechanism coupled to said shaft section and arranged to cause the tensive and/or compressive forces needed to articulate said bendable section to be applied to said bendable section.

2. The medical device of claim 1, wherein said device includes at least two of said slat members, each of which is fixed at one end to the distal end of said bendable section, said tensive and/or compressive forces applied to said bendable section via the counter-motion of said slat members.

3. The medical device of claim 1, wherein at least one of said slat members is fixed at both ends with one end fixed to the distal end of said bendable section, further comprising a movable wire, cable and/or slat member fixed at one end to the distal end of said bendable section such that said bendable section is articulated in the first plane by moving the wire, cable or slat with respect to the slat members which are fixed at both ends.

4. The medical device of claim 1, further comprising a wire, cable and/or slat member which is fixed at both ends with one end fixed to the distal end of said bendable section, wherein at least one of said slat members is movable and is fixed at one end to the distal end of said bendable section such that said bendable section is articulated in the first plane by moving said movable slats with respect to said fixed wire, cable and/or slat member.

5. A medical device with an articulating shaft assembly, comprising:
at least one slat member, each of said slat members having a predetermined cross section which allows the slat member to be substantially flexible in a first plane and relatively stiff in the plane perpendicular to said first plane;
a bendable section located at the working end of said device, said bendable section including at least a portion of at least one of said slat members and arranged to articulate in said first plane in response to the movement of said slat members;
a shaft section which is coupled to said bendable section and is sufficiently stiff to support said bendable section; and
at least one control mechanism coupled to said shaft section for moving said slat members and thereby causing said bendable section to articulate.

6. The device of claim 5, further comprising a handle attached to the end of said shaft section opposite said working end, said handle including said at least one control mechanism.

7. The device of claim 5, wherein each of said slat members has a height-to-thickness ratio of at least two-to-one.

8. The device of claim 5, wherein each of said slat members is comprised of a super-elastic or shape-memory material.

9. The device of claim 5, wherein each of said slat members is comprised of Nitinol.

10. The device of claim 5, wherein each of said slat members is threaded through the length of said bendable section.

11. The device of claim 5, wherein said bendable section includes means arranged to support and guide said slat members threaded through the length of said bendable section.

12. The device of claim 11, wherein said means comprises one or more slat channels through which respective ones of said slat members are threaded.

13. The device of claim 12, wherein said slat members are coated with a lubricous material to reduce or eliminate adhesion of said slat members to said slat channels.

14. The device of claim 12, further comprising at least one working channel which traverses said bendable section.

15. The device of claim 14, wherein said bendable section has an associated centerline and one of said working channels lies along said centerline and said slat channels lie off of said centerline.

16. The device of claim 14, further comprising an end effector coupled to the working end of said device, said device arranged such that said end effector can be operated via one or more of said working channels.

17. The device of claim 16, wherein at least one of said slat members is coupled to said end effector via respective channels and arranged to manipulate said end effector when pushed or pulled.

18. The device of claim 16, further comprising one or more wires which pass through said shaft and bendable sections via respective channels and are coupled to said end effector, said wires and end effector arranged such that said wires operate said end effector when pushed or pulled.

19. The device of claim 16, further comprising one or more wires which pass through said shaft and bendable sections via respective channels and are coupled to said end effector, said wires and end effector arranged such that said wires operate said end effector when pushed or pulled, and one or more slat members coupled to said end effector via respective channels and arranged to manipulate said end effector when pushed or pulled.

20. The device of claim 5, wherein said bendable section consists of materials of various durometers.

21. The device of claim 20, wherein said bendable section comprises a portion having a high durometer and a portion having a low durometer, said bendable section arranged such that, when articulated, said low durometer portion is stretched or compressed.

22. The device of claim 5, wherein said bendable section comprises an extrusion made from one or more plastic materials selected from a group consisting of silicone, thermoplastic elastomers (TPE), polyurethane, rubbers or plastics.

23. The device of claim 22, wherein said extruded plastic includes notches in the sides of said bendable section to increase its flexibility.

24. The device of claim 5, wherein said bendable section comprises a spring.

25. The device of claim 5, wherein each of said slat members is threaded through the length of said shaft section and said bendable section.

26. The device of claim 5, wherein said at least one control mechanism is coupled to said shaft section at the proximal end of said device and said bendable section is coupled to said shaft section at the distal end of said device.

27. The device of claim 5, further comprising a means for securing the terminal ends of said slat members to the distal end of said bendable section.

28. The device of claim 5, further comprising an end effector coupled to the working end of said device.

29. The device of claim 5, wherein said bendable section is at the distal end of said device and the articulation of said bendable section is controlled by the counter-motion of a second bendable section located at the proximal end of said device.

30. The device of claim 5, wherein said bendable section is in a ‘neutral’ state when said control mechanism is not imparting any force to cause said bendable section to articulate and in an ‘active’ state when said control mechanism is imparting force to cause said bendable section to articulate, said bendable section being substantially in-line with said shaft section when in said neutral state and bent with respect to said shaft section when in said active state.

31. The device of claim 5, wherein said bendable section is in a ‘neutral’ state when said control mechanism is not imparting any force to cause said bendable section to articulate and in an ‘active’ state when said control mechanism is imparting force to cause said bendable section to articulate, said bendable section being bent at a first angle with respect to said shaft section when in said neutral state and substantially in-line with said shaft section or at an angle other than said first angle when in said active state.

32. The device of claim 5, wherein said bendable section comprises two half sections arranged to interlock longitudinally to form a tube, the tips of said half sections at the working end of said bendable section pinned or fixed relative to each other such that said bendable section articulates when said half sections are moved in opposition to one another, such that said half sections function as said slat members.

33. A medical device with an articulating shaft assembly, comprising:
at least one slat member, each of said slat members having a predetermined cross section which allows the slat member to be substantially flexible in a first plane and relatively stiff in the plane perpendicular to said first plane;
a bendable section located at the working end of said device, said bendable section comprising:
one or more slat channels which traverse the length of said bendable section through which respective ones of said slat members are threaded, said bendable section arranged to articulate in response to the movement of said slat members;
at least one working channel which traverses the length of said bendable section, said bendable section having an associated centerline, said working channel arranged such that it lies along said centerline and said slat channels arranged such that they lie off of said centerline; and
a means for securing the terminal ends of said slat members to the distal end of said bendable section;
a shaft section which is coupled to said bendable section and is sufficiently stiff to support said bendable section; and
at least one control mechanism coupled to said shaft section for moving said slat members and thereby causing said bendable section to articulate.

34. The device of claim 33, further comprising an end effector coupled to the working end of said device, said device arranged such that said end effector can be operated via said working channel.
